(19) United States
(12) Patent Application Publication
(10) Pub. No.: US 2024/0423882 A1
(43) Pub. Date: Dec. 26, 2024

(54) A PERSONAL CLEANSING COMPOSITION

(71) Applicant: Conopco, Inc., d/b/a UNILEVER, Englewood Cliffs, NJ (US)

(72) Inventors: Praful Gulab Rao LAHORKAR, Bangalore (IN); Rajkumar PERUMAL, Bangalore (IN); Ashish Anant VAIDYA, Bangalore (IN)

(73) Assignee: Conopco, Inc., d/b/a UNILEVER, Englewood Cliffs, NJ (US)

(21) Appl. No.: 18/708,238

(22) PCT Filed: Nov. 8, 2022
(86) PCT No.: PCT/EP2022/081069

§ 371 Date: May 8, 2024

(30) Foreign Application Priority Data
Nov. 17, 2021 (EP) 21208863.7

(51) Int. Cl. A61K 8/19; A61Q 17/04; A61Q 19/10

(52) U.S. Cl. CPC A61K 8/19; A61Q 17/04; A61Q 19/10; A61K 2800/59

(57) ABSTRACT

The present invention relates to a personal cleansing composition that provides improved SPF. Particularly, the composition comprises water-soluble UVA sunscreen, water-soluble UVB sunscreen; and an alkali. The invention also relates to a method of and use for providing improved SPF.

### FIELD OF THE INVENTION

The present invention relates to a personal cleansing composition. Particularly, it relates to a personal cleansing composition that provides improved sun protection factor (SPF).

### BACKGROUND OF THE INVENTION

People often try to take care of themselves and of their external surfaces e.g. skin and scalp including hairs, with a desire of enjoying healthy lifestyle. Typically, people desire healthy and infection-free skin, good skin tone, adequate moisturization and protection from ultraviolet (UV) radiation contained in sunlight.

Sunlight comprises infrared region (700 nm to 1 mm), visible region (380 to 780 nm) and UV region (100 to 400 nm). The UV region is composed of UVC (100 to 280 nm), UVB (280 to 315 nm) and UVA (315 to 400 nm). While the human body needs some UV radiation for synthesis/maintenance of adequate amount of vitamin D, harmful effects of UV typically outweigh the benefits. An excessive exposure to UVB is said to cause direct damage to DNA and cause sunburn too; and an excessive exposure to UVA is said to give rise to immediate pigment darkening and delayed tanning effect. In addition, UVA is also said to cause DNA damages via oxidation reactions that involve reactive oxygen species (Zhang et al., 1997, Free Radical Biology & Medicine, 23, 980-985).

It is for reasons like these, people tend to avoid exposure to sunlight as much as possible. However, in many instances, avoiding exposure to sunlight is very difficult and at times; impossible. It is for such unavoidable reasons, people tend to make use of creams, lotions and gels comprising actives e.g. sunscreens, that offer at least some protection against unwanted effects caused by excessive exposure to UV radiations contained in sunlight. Sunscreens absorb UV radiations and prevent them from reaching a surface e.g. skin of a user, on to which it is applied. The ability of a sunscreen to provide protection by absorbing UVA radiation is expressed in terms of UVA protection factor (UVAPF); and the ability of a sunscreen to provide protection by absorbing UVB radiation is expressed in terms of sun protection factor (SPF).

Such leave-on compositions comprising sunscreens are generally applied on to the skin before getting exposed to sunlight. A problem with such leave-on compositions is such that its application is time consuming. Additionally, leave-on compositions are generally delivered through a cream, a lotion and a gel. Some consumers tend to dislike the sensorial properties of such products and/or dislike sticky or oily feel such leave-on compositions may leave behind.

On the other hand, delivering actives e.g. sunscreens, on to the skin through personal cleansing or wash-off compositions e.g. face wash and body wash, is an extremely difficult challenge as they tend to get washed off in the process of bathing or washing the skin. Thus, delivering an enhanced deposition of actives e.g. sunscreens, through personal cleansing compositions continues to be a challenge.

Need therefore exists to provide a personal cleansing composition comprising active e.g. sunscreens that addresses the problem.

It has now been found that a personal cleansing composition comprising select water soluble UVA sunscreen, select water soluble UVB sunscreens and one or more select alkali, resulted in higher deposition of these sunscreens thereby providing improved SPF.

### SUMMARY OF THE INVENTION

In a first aspect, the present invention relates to a personal cleansing composition comprising:
a. 0.1 to 10 wt % water-soluble UVA sunscreen,
b. 0.1 to 10 wt % water-soluble UVB sunscreen,
c. 0.01 to 6 wt % an alkali selected from calcium hydroxide, magnesium hydroxide, beryllium hydroxide and mixtures thereof,
wherein the solubility of the UV-A or UV-B sunscreen in water is higher than 50 g/L at 25° C.

In a second aspect, the present invention relates to a method of providing improved sun protection factor comprising steps:
i. applying the composition of the first aspect on to a surface; and
ii. removing the composition from the surface after a duration ranging from 10 seconds to 5 minutes,
wherein the sun protection factor is at least 5 and maximum 50.

In a third aspect, the present invention relates to use of the composition of the first aspect for providing improved sun protection factor wherein the sun protection factor is at least 5 and maximum 50.

In a fourth aspect, the present invention relates to use of an alkali in a personal cleansing composition to improve the sunscreen protection factor of the composition, wherein the alkali is selected from calcium hydroxide, magnesium hydroxide, beryllium hydroxide and mixtures thereof, wherein the personal cleansing composition further comprises 0.1 to 10 wt % water-soluble UVA sunscreen; and 0.1 to 10 wt % water-soluble UVB sunscreen; and wherein the sun protection factor is at least 5 and maximum 50.

### DETAILED DESCRIPTION OF THE INVENTION

Any feature of one aspect of the present invention may be utilized in any other aspect of the invention. The word “comprising” is intended to mean “including” but not necessarily “consisting of” or “composed of.” In other words, the listed steps or options need not be exhaustive. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “x to y”, it is understood that all ranges combining the different endpoints are also contemplated. Unless specified otherwise, amounts as used herein are expressed in percentage by weight based on total weight of the composition and is abbreviated as “wt %”. The use of any and all examples or exemplary language e.g. “such as” provided herein is intended merely to better illuminate the invention and does not in any way limit the scope of the invention otherwise claimed.

### A Personal Cleansing Composition

For the purpose of the present invention, the phrases “a personal cleansing composition” and “a wash-off composition”, “a rinse-off composition” may be used interchangeably and the denote the same meaning. By “a personal cleansing composition” or “a wash-off composition” or a “rinse-off composition” as used herein, is meant to include a composition for cleaning topical surfaces of mammals, especially humans. This composition is particularly useful for use on the sun-exposed parts of the body. Such a composition is generally of the rinse off type which typically means that the composition is high in surfactants which are known to help in cleaning surfaces to make them free of oils and dirt. The composition is generally used by diluting with water as it is applied on to skin, scalp and hair, after which the user works up a lather to ensure that the dirt and oil on the surface are removed and the surface is then rinsed with copious amounts of water to ensure that the composition is removed from the body surface it was applied on to. The composition of the present invention is preferably in the form of a liquid e.g. a hand wash, a body wash and a shampoo.

“Skin” as used herein is meant to include skin on the face and body (e.g. neck, chest, back, arms, underarms, hands, legs, buttocks and scalp) and especially to the sun exposed parts thereof. The composition of the invention is also of relevance to application on any other keratinous substrates of the human body other than skin e.g. scalp and hair, where products may be formulated with specific aim of providing protection from UV radiations.

A personal cleansing composition according to the present invention (the composition) comprises a water-soluble UVA sunscreen, a water-soluble UVB sunscreen; and an alkali selected from calcium hydroxide, magnesium hydroxide, beryllium hydroxide and mixtures thereof.

### Water Soluble UVA Sunscreen

The composition comprises a water soluble UVA Sunscreen. Preferably, ‘water soluble’ means that solubility in water is higher than 50 g/L at 25° C.

Preferably, the water soluble UVA sunscreen is selected from di sodium phenyl dibenzimidazole tetra sulfonate, terephthalylidene dicamphor sulfonic acid and mixtures thereof.

The composition comprises 0.1 to 10 wt %, preferably 0.1 to 8 wt %, more preferably 0.5 to 6 wt %, even more preferably 1 to 5 wt %, further more preferably 1 to 4 wt %, still more preferably 1 to 3 wt % and yet more preferably 1 to 2 wt % water soluble UVA sunscreen. Water soluble UVB Sunscreen

The composition comprises water soluble UVB sunscreens. Preferably, ‘water soluble’ means that solubility in water is higher than 50 g/L at 25° C.

Preferably, the water-soluble UVB sunscreen is selected from phenyl benzimidazole sulphonic acid (PBSA), benzylidene camphor sulfonic acid, benzophenone-4 and mixtures thereof.

The composition comprises 0.1 to 10 wt %, preferably 0.1 to 8 wt %, more preferably 0.5 to 6 wt %, even more preferably 1 to 5 wt %, further more preferably 1 to 4 wt %, still more preferably 1 to 3 wt % and yet more preferably 1 to 2 wt % water soluble UVB sunscreen. Alkali

The composition comprises an alkali selected from calcium hydroxide, magnesium hydroxide, beryllium hydroxide and mixtures thereof. Preferably, the alkali is selected from calcium hydroxide, magnesium hydroxide and mixtures thereof. More preferably, the alkali is calcium hydroxide.

Preferably, an alkali needs to be free to form a complex with an acid e.g. phenyl benzimidazole sulfonic acid (one of the water soluble UVB sunscreens as per the present invention). That said, an alkali e.g. aluminium hydroxide complexed with other substances e.g. titanium dioxide and stearic acid, like the commercially available products e.g. MT 100Z and MT100TV, is not suitable for the purpose of the present invention.

The composition comprises 0.01 to 6 wt %, preferably 0.05 to 5 wt %, more preferably 0.1 to 5 wt %, even more preferably 0.5 to 4 wt %, further more preferably 1 to 3 wt % and still more preferably 1 to 2 wt % of the alkali.

It will be understood that if more than one alkali as mentioned above may be present in the composition, their collective weight percentage will be in the ranges as described above. Nonionic surfactant

Preferably, the composition further comprises 5 to 40 wt % nonionic surfactant. Preferably, the nonionic surfactant is selected from nonionic surfactants having HLB values 7 to 20 more preferably 9 to 20, more preferably 10 to 19, even more preferably 12 to 18, even more preferably 13 to 17 and still more preferably 15 to 17.

HLB is calculated using the Griffin method wherein HLB=20×Mh/M wherein Mh is the molecular mass of the hydrophilic portion of the molecule and M is the molecular mass of the whole molecule, giving a result on an arbitrary scale of 0 to 20. Typical values for various surfactants are given below:
A value <10: Lipid soluble (water insoluble),
a value >10: Water soluble,
a value from 4 to 8 indicates an anti-foaming agent,
a value from 7 to 11 indicates a W/O (water in oil) emulsifier,
a value from 12 to 16 indicates oil in water emulsifier,
a value from 11 to 14 indicates a wetting agent,
a value from 12 to 15 is typical of detergents,
a value of 16 to 20 indicates a solubiliser or a hydrotrope.

Preferably, the nonionic surfactant having HLB value in the range 7 to 20 is selected from fatty alcohol ethoxylates, alkyl phenol ethoxylates, polyoxyethylene sorbitan alkyl esters, alkyl polyglucoside and mixtures thereof. Preferably, the nonionic surfactants are ones with at least 9 alkylene oxide groups e.g. 9 ethylene oxide groups.

Examples of fatty alcohol ethoxylates that may be used as nonionic surfactants in the composition include polyoxyethylene lauryl ether (HLB=16.9; commercially available as Brij® 35), polyoxyethylene (20) cetyl ether (HLB=16; commercially available as Brij® 58), polyethylene glycol octadecyl ether (HLB=18.8; commercially available as Brij® 700) and Laureth-9 (C12EO9; HLB=14.3; commercially available as Brij® L9).

Examples of alkyl phenol ethoxylates that may be used as nonionic surfactant in the composition include octylphenol ethoxylate (HLB=15.5; commercially available as Triton™ X165), octylphenol ethoxylate (HLB=17.6; commercially available as Triton™ X405) and octylphenol ethoxylate (HLB=18.4; commercially available as Triton™ X705).

Examples of polyoxyethylene sorbitan alkyl esters that may be used as the nonionic surfactant in the composition include polyoxyethylenesorbitan monolaurate (HLB=13.3; commercially available as Tween® 21), polyoxyethylenesorbitan monolaurate (HLB=16.7; commercially available as Tween® 20), Polyoxyethylenesorbitan monopalmitate (HLB=15.6; commercially available as Tween® 40) and polyoxyethylene sorbitan monostearate (HLB=14.9; commercially available as Tween® 60).

Examples of alkyl polyglucoside that may be used as the nonionic surfactant in the composition include lauryl glucoside (HLB =11.6; commercially available as PLANTACARE® 1200 UP), decyl glucoside (HLB =12.8; commercially available as PLANTACARE® 2000 UP), caprylyl/capryl glucoside (HLB =13.4; commercially available as PLANTACARE® 810 UP), coco-glucoside (HLB =13.1; commercially available as PLANTACARE® 818 UP),

More preferably, the nonionic surfactant having HLB value in the range 9 to 20 that may be present in the composition is fatty alcohol ethoxylate with saturated carbon chain having HLB higher than 15.5.

Preferably, the composition comprises 5 to 40 wt %, more preferably 7 to 35 wt %, even more preferably 10 to 30 wt %, further more preferably 12 to 28 wt %, still more preferably 15 to 25 wt %, yet more preferably 18 to 25 wt % and still further more preferably 20 to 23 wt % of the nonionic surfactant.

### Anionic Surfactant

Preferably, the composition comprises an anionic surfactant. The anionic surfactant may be selected from primary alkyl sulphates, ethoxylated alkyl sulphates and combinations thereof. Preferred alkyl sulphates are C8-18 alky sulfates, more preferably C12-18 alkyl sulphates, preferably in the form of a salt with a solubilising cation such as sodium, potassium, ammonium or substituted ammonium. Preferred ethoxylated alkyl sulphates are those having the formula: RO(CH₂CH₂O)ₙSO₃M; wherein R is an alkyl or alkenyl having 8 to 18 (preferably 12 to 18) carbon atoms; n is a number having an average value of greater than at least 0.5, preferably between 1 and 3, more preferably between 2 and 3; and M is a solubilising cation such as sodium, potassium, ammonium or substituted ammonium. An example is sodium lauryl ether sulphate (SLES). SLES having an average degree of ethoxylation of from 0.5 to 3, preferably 1 to 3 is especially preferred.

Preferably, the composition comprises 0 to 5 wt %, more preferably 1 to 4 wt % and further more preferably 2 to 3 wt % anionic surfactant.

### Cationic Surfactant

Preferably, the composition comprises a cationic surfactant. A cationic anionic surfactant may be selected from stearamidopropyl dimethylamine, behentrimonium chloride, or stearyl trimethyl ammonium chloride, Dimethyldioctadecylammonium bromide, N,N-Dihexadecyl-N-methyl-1-hexadecanaminium chloride, dicoco dimethyl ammonium chloride, Behenyl Trimethyl Ammonium Chlorid, alkyl dimethyl benzyl ammonium chloride, benzalkonium chloride, didecyl dimethyl ammonium chloride, cetrimide or dodecyltrimethylammonium chloride. Such surfactants are commercially available.

Preferably, the composition comprises 0 to 5 wt %, preferably 1 to 4 wt % and more preferably 2 to 3 wt % cationic surfactant.

### Amphoteric Surfactant

Preferably, the composition comprises an amphoteric surfactant as a co-surfactant along with either a nonionic surfactant or an anionic surfactant. An amphoteric surfactant may be selected from cocoamidopropyl betaine, lauryl betaine, betaines, coco alkyldimethyl, (carboxymethyl)dimethyloleylammonium hydroxide or (carboxylatomethyl)dimethyl(octadecyl)ammonium.

Preferably, the composition comprises 0 to 15 wt %, more preferably 1 to 13 wt %, even more preferably 1 to 10 wt %, further more preferably 1 to 8 wt %, still more preferably 1 to 5 wt % and yet more preferably 1 to 3 wt % amphoteric surfactant.

### pH of the Composition

Preferably, if the compositions is formulated in the form a liquid composition, then the pH of the composition is in the range 6.5 to 8.5, more preferably 7.0 to 8.5, even more preferably 7.0 to 7.5.

### Water

Preferably, the composition comprises 5 to 90 wt % water. The amount of water contained in the composition depends on the final product format. For example, if the composition is in the form of a liquid composition, then preferably 50 to 80 wt %, more preferably 55 to 75 wt %, even more preferably 60 to 70 wt % water may be present in the composition.

### Silicone Oils

Preferably, the composition comprises 0 to 5 wt %, more preferably 0.1 to 4 wt % and even more preferably 1 to 3 wt % silicone oils. Preferably, silicone oil may be added to confer better sensory properties to the composition. For example, 1 wt % silicone oil may be added to the composition if it is to be formulated as shampoo and face wash. A silicone oil may be selected from polyalkyl siloxanes, polyaryl siloxanes, polyalkylaryl siloxanes, polyether siloxane copolymers and mixtures thereof. Another useful type are the crosslinked silicone elastomers such as Dimethicone/Vinyl/Dimethicone Crosspolymers (e.g. Dow Corning 9040 and 9041). If the composition is formulated as a shampoo composition, then amino silicones may also be formulated therein. Amino silicones are silicones containing at least one primary amine, secondary amine, tertiary amine or a quaternary ammonium group.

Further, the composition may preferably comprise a polyhydric alcohol (also called polyol) or mixture of polyols. Polyol is a term used herein to designate a compound having multiple hydroxyl groups (at least two, preferably at least three) which is highly water soluble. Many types of polyols are available including relatively low molecular weight short chain polyhydroxy compounds such as glycerol and propylene glycol; sugars such as sorbitol, manitol, sucrose and glucose; modified carbohydrates such as hydrolyzed starch, dextrin and maltodextrin, and polymeric synthetic polyols such as polyalkylene glycols, for example polyoxyethylene glycol (PEG) and polyoxypropylene glycol (PPG). Especially preferred polyols are glycerol, sorbitol and mixtures thereof. Most preferred polyol is glycerol.

Preferably, the composition comprises polymers. Polymers of the acrylate class are especially preferred. Examples of acrylate polymers include polymers and copolymers of acrylic acid crosslinked with polyallylsucrose as described in U.S. Pat. No. 2,798,053 which is herein incorporated by reference. Other examples include polyacrylates, acrylate copolymers or alkali swellable emulsion acrylate copolymers, hydrophobically modified alkali swellable copolymers, and crosslinked homopolymers of acrylic acid. Examples of such commercially available polymers are: ACULYN®, CARBOPOL®, and CARBOPOL® Ultrez grade series. [Carbopol® Aqua SF-1 polymer, INCI: Acrylates Copolymer supplied by Lubrizol].

Preferred use level in liquid cleanser formats 0.1 to 8%, preferably 1 to 6%, more preferably 1.5 to 4%, even more preferably 2 to 3%.

### Optional Ingredients

The composition may further comprise 0 to 15 wt %, more preferably 0 to 10 wt % and further more preferably 0 to 5 wt % optional ingredients selected from anti-oxidants, perfumes, chelating agents, colourants, deodorants, dyes, enzymes, foam boosters, germicides, anti-microbials, lathering agents, pearlescers, skin conditioners, stabilizers.

If the composition is formulated as a shampoo composition, then to further aid in deposition of actives, cationic polymers are generally included. Preferably, the shampoo composition includes. A cationic polymer is preferably guar hydroxypropyl trimonium chloride. Guar polymer predominantly contains galactomannan polymer chains. This polymer is available at various molecular weights and degree of cationic substitutions depending on how much the guar has been hydrolysed and cationised. Preferably, the composition comprises 0.01 to 2.0 wt %, more preferably 0.04 to 0.5 wt % and even more preferably 0.08 to 0.25 wt % cationic polymer.

In a second aspect, the invention relates to a method of providing improved sun protection factor comprising steps:
i. applying the composition of the first aspect on to a surface; and
ii. removing the composition from the surface after a duration ranging from 10 seconds to 5 minutes,
wherein the sun protection factor is at least 5 and maximum 50.

When the composition is applied as per the method described above, SPF of at least 5 and maximum 50 is obtained. Preferably, the method is non-therapeutic or cosmetic in nature.

In a third aspect, the present invention relates to a use of the composition of the first aspect for providing improved sun protection factor wherein the sun protection factor is at least 5 and maximum 50. Preferably, the use is non-therapeutic or cosmetic in nature.

Preferably, the SPF is at least 10 and maximum 50, more preferably at least 15 and maximum 50, even more preferably at least 20 and maximum 50.

In a fourth aspect, the present invention relates to use of an alkali in a personal cleansing composition to improve the sunscreen protection factor of the composition, wherein the alkali is selected from calcium hydroxide, magnesium hydroxide, beryllium hydroxide and mixtures thereof; and wherein the personal cleansing composition further comprises 0.1 to 10 wt % water-soluble UVA sunscreen; and 0.1 to 10 wt % water-soluble UVB sunscreen; and wherein the sun protection factor is at least 5 and maximum 50. Preferably, the alkali is used in an amount 0.01 to 6 wt %, more preferably 0.05 to 5 wt %, even more preferably 0.1 to 5 wt %, further more preferably 0.5 to 4 wt %, still more preferably 1 to 3 wt % and yet more preferably 1 to 2 wt %.

The invention is further described below by way of the following non-limiting examples:

### EXAMPLES

Briefly, compositions shown in tables below were prepared as follows:

Phase A was prepared where water soluble UV-A sunscreen and water soluble UV-B sunscreens and alkali as shown in tables below were combined in presence of water. Phase A was prepared at temperatures in the range 40 to 60° C. e.g. 50° C. On the other hand, phase B was prepared by mixing the other ingredients e.g. surfactant, thickener, polymer, polyols in water. Phase B was prepared in water at around 60° C. After this, phase A and phase B were mixed around 60° C. using a homogenizer and mixing was carried out for 10 to 15 minutes post which the mixture was cooled at room temperature; and used further in the experimentation.

### Measurement of In-Vitro SPF

The compositions were diluted with water and then applied on to pre-wet vitro skin. The vitro skin was then rinsed off immediately post product application and kept for drying in the dark. After drying, the vitro skin was exposed to UV light and transmittance scan was recorded. This scan gives the transmittance as a function of wavelength (290-400 nm) for a given sample. For a single vitro skin sample four different spots were scanned. The same was repeated for 3 vitro skin samples. The data reported is thus an average of 12 readings. The reference transmittance scan was obtained using blank vitro skin, with glycerine spread on it as control. The transmittance values were recorded using UV-2000S Ultraviolet Transmittance Analyzer. UV-2000S application software provided with the instrument gives the in vitro SPF based on the measured transmittance values.

TABLE 1
Composition according to the invention (example 1) and control (example A)

| Ingredients (in wt %) | A | 1 |
|---|---|---|
| Disodium Phenyl Dibenzimidazole Tetrasulfonate | 1.2 | 1.2 |
| (Water soluble UVA sunscreen; Neoheliopan ® AP; | | |
| Symrise) | | |
| Phenyl benzimidazole | 3 | 3 |
| sulphonic acid | | |
| (Water soluble UVB sunscreen; Ensulizole; Merck) | | |
| Potassium hydroxide | 1.01 | — |
| Calcium hydroxide | — | 0.584 |
| Decyl glycoside | 6 | 6 |
| Lauryl glycoside | 4 | 4 |
| Polysorbate-20 | 3 | 3 |
| Laureth-23 | 3 | 3 |
| Glycerol | 2 | 2 |
| Water | To 100 | To 100 |
| In-vitro SPF | 15.97 | 28.33 |
| S.D. | 3.67 | 6.30 |

Note:
SPF of blank (glycerol) was 1.00 ± 0.01

TABLE 2
Additional examples

| Ingredients (in wt %) | B | C | 2 | 3 | 4 | 5 |
|---|---|---|---|---|---|---|
| Disodium Phenyl | 1.20 | 1.20 | 1.20 | 1.20 | 1.20 | 1.20 |
| Dibenzimidazole Tetrasulfonate | | | | | | |
| (Water soluble UVA sunscreen; | | | | | | |
| Neoheliopan ®AP; Symrise) | | | | | | |
| Phenyl benzimidazole sulphonic | 3.00 | 3.00 | 3.00 | 3.00 | 3.00 | 3.00 |
| acid (Water soluble UVB | | | | | | |
| sunscreen; Ensulizole; Merck) | | | | | | |
| KOH | 1.010 | 1.010 | — | — | — | — |
| Ca(OH)₂ | — | — | 0.584 | 0.584 | — | 0.584 |
| Mg(OH)₂ | — | — | — | — | 0.464 | — |
| Decyl Glycoside | — | 6 | — | 6 | 6 | 6 |
| Lauryl Glycoside | — | 4 | — | 4 | 4 | 4 |
| Polysorbate 20 | — | 3 | — | 3 | 3 | 1 |
| Laureth-23 | — | 3 | — | 3 | 3 | 1 |
| SLES-1 EO (for 100%) | 12.00 | — | 12.00 | — | — | — |
| CAPB (30%) | 13.40 | — | 13.40 | — | — | 13.40 |
| Glycerol | 2.00 | 2.00 | 2.00 | 2.00 | 2.00 | 2.00 |
| Water | To 100 | To 100 | To 100 | To 100 | To 100 | To 100 |
| In vitro SPF | 18.51 | 15.86 | 19.71 | 32.37 | 27.47 | 23.81 |
| STDV | 0.29 | 2.30 | 0.82 | 4.92 | 2.53 | 2.51 |

1. A personal cleansing composition comprising:
a. 0.1 to 10 wt % water-soluble UVA sunscreen,
b. 0.1 to 10 wt % water-soluble UVB sunscreen,
c. 0.01 to 6 wt % an alkali selected from calcium hydroxide, magnesium hydroxide or mixtures thereof,
wherein the solubility of the UV-A or UV-B sunscreen in water is higher than 50 g/L at 25° C.; and
d. 5 to 40 wt % non-ionic surfactant.

2. (canceled)

3. The personal cleansing composition as claimed in claim 1, wherein the water-soluble UVA sunscreen is selected from di sodium phenyl dibenzimidazole tetra sulfonate, terephthalylidene dicamphor sulfonic acid and mixtures thereof.

4. The personal cleansing composition as claimed in claim 1, wherein the water-soluble UVB sunscreen is selected from phenyl benzimidazole sulphonic acid, benzylidene camphor sulfonic acid, benzophenone-4 and mixtures thereof.

5. The personal cleansing composition as claimed in claim 1, wherein the alkali is in its free form to form a complex with the sunscreen.

6. The personal cleansing composition as claimed in claim 1, wherein the alkali is calcium hydroxide.

7. The personal cleansing composition as claimed in claim 1, wherein the nonionic surfactant is selected from nonionic surfactants having HLB values from 7 to 20.

8. The personal cleansing composition as claimed in claim 1, wherein the composition further comprises from 0 to 5 wt % anionic surfactant.

9. The personal cleansing composition as claimed in claim 1, wherein the composition further comprises from 0 to 5 wt % cationic surfactant.

10. The personal cleansing composition as claimed in claim 1, wherein the composition further comprises from 0 to 15 wt % amphoteric surfactant.

11. The personal cleansing composition as claimed in claim 1, wherein the pH of the composition is in the range from 6.5 to 8.5.

12. The personal cleansing composition as claimed in claim 1, wherein the composition comprises 5 to 90 wt % water.

13. A method of providing improved sun protection factor comprising steps:
i. applying the personal cleansing composition as claimed in claim 1 on to a surface; and
removing the composition from the surface after a duration ranging from 10 seconds to 5 minutes,
wherein the sun protection factor is at least 5 and maximum 50.

14. The personal cleansing composition as claimed in claim 1, wherein the personal cleansing composition for providing provides an improved sun protection factor, wherein the sun protection factor is at least 5 and maximum 50.

15. A personal cleansing composition comprising an alkali to improve the sunscreen protection factor of the composition, wherein the alkali is selected from calcium hydroxide, magnesium hydroxide or mixtures thereof; and wherein the personal cleansing composition further comprises 0.1 to 10 wt % water-soluble UVA sunscreen; and 0.1 to 10 wt % water-soluble UVB sunscreen; and 5 to 40 wt % non-ionic surfactant;
wherein the sun protection factor is at least 5 and maximum 50.
